(19) United States
(12) Patent Application Publication
(10) Pub. No.: US 2007/0281957 A1
(43) Pub. Date: Dec. 6, 2007

(54) Crystalline Forms of Levorphanol

(75) Inventors: Sidney Liang, Olivette, MO (US); Gary A. Nichols, Wildwood, MO (US); Michelle R. Menze, St. Louis, MO (US)

Correspondence Address: Mallinckrodt Inc., 675 McDonnell Boulevard, HAZELWOOD, MO, 63042, US

(73) Assignee: Mallinckrodt Inc., Hazelwood, MO (US)

(21) Appl. No.: 11/753,012

(22) Filed: May 24, 2007

Related U.S. Application Data
(60) Provisional application No. 60/810,975, filed on Jun. 5, 2006.

(51) Int. Cl. A61K 31/485; C07D 489/02

(52) U.S. Cl. 514/282; 546/44

(57) ABSTRACT

The present invention relates to novel crystalline polymorphic forms of levorphanol including hydrated, solvated, and non-hydrated (non-solvated) forms. The invention also describes methods of preparing the various polymorphic forms. The present invention also relates to pharmaceutical compositions containing crystalline polymorphs of levorphanol, as well as methods of relieving pain by administering the pharmaceutical compositions.

### CROSS REFERENCE TO RELATED APPLICATIONS

This application claims the benefit of the filing date of U.S. Provisional Application Ser. No. 60/810,975, filed Jun. 5, 2006, entitled “Crystalline Forms of Levorphanol” which is incorporated herein in their entirety.

### BACKGROUND OF THE INVENTION

1. Field of the Invention

The present invention relates to novel crystalline polymorphic forms of levorphanol, compositions containing them, and methods for making polymorphic forms of levorphanol. These novel polymorphs of levorphanol have activity over a wide range of indications, and are particularly useful for the treatment of pain. The invention also encompasses related processes, compositions, and methods.

2. Background of the Invention

Levorphanol (3-Hydroxy-N-methylmorphinan; CAS No. 77-07-6) is a well-known narcotic analgesic. It has a molecular formula C₁₇H₂₃NO, a molecular weight of 257.38 g/mol. and the following structural formula:

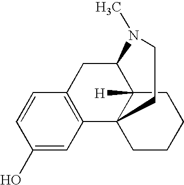

Levorphanol free base is known to have the following physical properties:

| Property | Levorphanol |
|---|---|
| Melting point | 198° C.–199° C. |
| Water solubility (at 25° C.) | 1840 mg/L |
| Vapor pressure (at 25° C.) | 1.04 × 10⁻⁶ mmHg |
| pKa | 9.58 |

The most common salt of levorphanol is levorphanol tartrate (CAS No. 125-74-6)

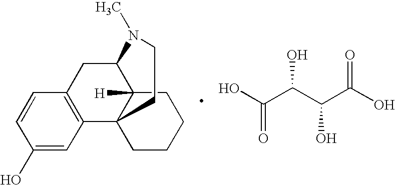

Levorphanol and its preparation are described in many patents and publications including, for example, U.S. Pat. No. 2,524,855; U.S. Pat. No. 2,638,472; U.S. Pat. No. 2,769,810; U.S. Pat. No. 3,085,091; U.S. Pat. No. 3,211,738; Schneider et al., “Hydroxymorphinan. V. Optically active benzyloctahydroisoquinolines” Helv. Chim. Acta 37, 710-720, 1954; DE 802,571 (“3-Hydroxy-N-methylmorphinan and its salts”); and Schneider et al., “Synthesis of morphinans” Helv. Chim. Acta 33, 1437-1448, 1950. None of these references describe the synthesis of polymorphic forms of levorphanol.

In accordance with the present invention, several novel polymorphic forms of levorphanol are provided, as well as methods for synthesizing them. The polymorphs of levorphanol described herein are particularly promising for use in the management of pain. The present invention fulfills the need in the art for additional compositions and methods for treating pain, and provides further related advantages.

### SUMMARY OF THE INVENTION

The present invention is directed to crystalline polymorphs, Forms I-X, as well as mixtures thereof. The present invention further pertains to the use of these crystalline forms in the treatment of pain, and to pharmaceutical formulations containing them.

One aspect of the invention is directed to polymorphic forms of levorphanol.

A second aspect of the invention is directed to non-solvated polymorphic forms of levorphanol, more particularly, non-hydrated polymorphic forms of levorphanol.

A third aspect of the invention is directed to solvated polymorphic forms of levorphanol, in particular, acetonitrile solvated forms of levorphanol.

A fourth aspect of the invention is directed to solvated polymorphic forms of levorphanol, in particular, methylene chloride solvated forms of levorphanol.

A fifth aspect of the invention is directed to solvated polymorphic forms of levorphanol, in particular, chloroform solvated forms of levorphanol.

A sixth aspect of the invention is directed to solvated polymorphic forms of levorphanol, in particular, alcohol solvated forms of levorphanol.

A seventh aspect of the invention is directed to hydrated polymorphic forms of levorphanol.

Other novel features and advantages of the present invention will become more apparent to those skilled in the art upon examination of the following or upon learning by practice of the invention.

### BRIEF DESCRIPTION OF THE FIGURES

FIG. 1 shows the powder X-ray diffraction (pXRD) pattern for a new polymorph of levorphanol—Form I.

FIG. 2 shows the powder X-ray diffraction (pXRD) pattern for a new polymorph of levorphanol—Form II.

FIG. 3 shows the powder X-ray diffraction (pXRD) pattern for a new polymorph of levorphanol—Form III.

FIG. 4 shows the powder X-ray diffraction (pXRD) pattern for a new polymorph of levorphanol—Form IV.

FIG. 5 shows the powder X-ray diffraction (pXRD) pattern for a new polymorph of levorphanol—Form V.

FIG. 6 shows the powder X-ray diffraction (pXRD) pattern for a new polymorph of levorphanol—Form VI.

FIG. 7 shows the powder X-ray diffraction (pXRD) pattern for a new polymorph of levorphanol—Form VII.

FIG. 8 shows the powder X-ray diffraction (pXRD) pattern for a new polymorph of levorphanol—Form VIII.

FIG. 9 shows the powder X-ray diffraction (pXRD) pattern for a new polymorph of levorphanol—Form IX.

FIG. 10 shows the powder X-ray diffraction (pXRD) pattern for a new polymorph of levorphanol—Form X.

### DETAILED DESCRIPTION OF THE INVENTION

The present invention describes several novel polymorphic forms of levorphanol, and methods of making them.

### Polymorph Production

Several crystalline forms of levorphanol were prepared and subsequently characterized. These crystalline forms include three non-solvated/non-hydrated polymorphs (herein denoted Forms I, II, and IX), an acetonitrile solvate (Form III), a methylene chloride solvate (Form IV), two chloroform solvates (Forms V and VI), a methyl alcohol solvate (Form VII), and hydrated polymorphs (Forms VIII and X). The methods used to produce each of the crystalline forms from levorphanol base and/or levorphanol tartrate are set forth in Table 1. Each crystalline form exhibited a distinctly different powder X-ray diffraction pattern.

TABLE 1
Production of Levorphanol Hydromorphs

| Form | Description | Preparation |
|---|---|---|
| I | Non-Solvated/Non- | Obtained by crystallization from ethyl alcohol, isopropyl |
| | Hydrated | alcohol, and methyl alcohol/water mixtures. |
| | | Obtained by slurrying Levorphanol Base in methyl |
| | | alcohol and methyl alcohol/water mixtures. |
| | | Obtained when Levorphanol Base is sublimed. |
| II | Non-Solvated/Non- | Obtained by crystallization from ethyl ether, |
| | Hydrated | hexane/acetone mixtures, toluene, and water/methyl |
| | | alcohol mixtures. |
| | | Obtained by drying the bound solvent from samples |
| | | present as Form IV, V, & VI. |
| | | Obtained by neutralization of Levorphanol Tartrate |
| | | Dihydrate with NH₄OH in water. |
| III | Acetonitrile Solvate | Obtained by crystallization from acetonitrile. |
| | | Obtained by slurrying Levorphanol Base in acetonitrile. |
| IV | Methylene Chloride | Obtained by crystallization from methylene chloride. |
| | Solvate | Obtained by slurrying Levorphanol Base in methylene |
| | | chloride. |
| V | Chloroform Solvate | Obtained by slurrying Levorphanol Base in chloroform. |
| VI | Chloroform Solvate | Obtained by crystallization from chloroform. |
| | | Obtained by slurrying Levorphanol Base in chloroform. |
| VII | Methyl Alcohol Solvate | Obtained by crystallization from methyl alcohol. |
| VIII | Contains Bound Water | Obtained by neutralization of Levorphanol Tartrate |
| | (~2.0% water by mass) | Dihydrate with NH₄OH in water. |
| IX | Non-Solvated/Non- | Obtained by drying a sample of Form VIII at 105° C. |
| | Hydrated | |
| X | Monohydrate | Obtained by crystallization from acetone/water |
| | | mixtures. |

### Polymorph Characterization

### Differential Scanning Calorimetry

A TA Instruments Q100—differential scanning calorimeter was used. The samples were weighed into an aluminum, hermetic sample pan and were crimped with a pinhole lid. The samples were heated from 25° C. to 220° C. at a rate of 10° C. per minute (unless otherwise noted).

Form I was discovered to melt/decompose at a temperature of approximately 200° C. by DSC and hot-stage microscopy, while Form II was observed to first melt/recrystallize from 160° C. to 180° C., and then melt/decompose at approximately 200° C.

Form II exhibited a series of phase transitions (melt and recrystallization—conversion of one crystalline form to another) from approximately 160° C. to 180° C. by DSC. Different scanning rates were observed to have a dramatic effect on these transitions. Form II exhibited an exothermic transition likely associated with recrystallization (following desolvation) by DSC. The sample appeared to melt/decompose at approximately 200° C. These transitions were confirmed by hot-stage microscopy.

Form III exhibited a broad endothermic transition from 80-145° C., and was discovered to melt/decompose at approximately 200° C.

Form IV exhibited consecutive endothermic/exothermic transitions from 100-170° C. Final melting/decomposition occurred in an endothermic transition at approximately 200° C.

Form V exhibited several overlapping endothermic/exothermic transitions from 60-145° C. Final melting/decomposition occurred in an endothermic transition from 175-200° C.

Form VI exhibited two endothermic transitions from 100-150° C. Final melting/decomposition occurred in an endothermic transition at approximately 200° C.

Form VII exhibited a small endothermic transition from 145-155° C. Final melting/decomposition occurred in an endothermic transition at approximately 200° C.

Form VIII exhibited two exothermic transitions subsequent to desolvation, including a broad endothermic transition from 30-100° C., and a small exothermic transition from 145-155° C. Final melting/decomposition occurred in an endothermic transition at approximately 200° C.

Form IX exhibited an exothermic transition from 145-155° C. Final melting/decomposition occurred in an endothermic transition at approximately 200° C.

For Form X, no DSC data was collected.

### Thermogravimetric Analysis and TGA-Fourier Transform Infrared Spectroscopy

A TA Instruments Q50—thermogravimetric analyzer equipped with a quartz lined evolved gas furnace was used to conduct thermogravimetric analysis-Fourier transform infrared spectroscopy (TGA-FTIR). The furnace was coupled to a Nicolet Nexus 470 equipped with a TGA interface furnace, gas cell, and transfer line. The samples were heated from 10° C. per minute to approximately 200° C. (unless otherwise noted). The transfer line and TGA interface furnace were held at 150° C. A total nitrogen flow rate of 50 mL/min was used for all experiments. A Gram-Schmidt plot/analysis was attained for the experiments, with individual spectra of evolved gases analyzed as follows: 16 scans, 8 cm⁻¹. A background (16 scans) was acquired prior to analyses.

Form I and Form II exhibited no loss of mass associated with a bound solvent by TGA, prior to sublimation above 135° C.

Form III exhibited 6-7% loss of mass from 80-145° C. The lost material was identified as acetonitrile by TGA-FTIR. This sample is therefore present, at least in part, as an acetonitrile solvate.

Form IV exhibited a 7-8% loss of mass from 110-140° C. The lost material was identified as methylene chloride by TGA-FTIR. This sample is therefore present, at least in part, as a methylene chloride solvate.

Form V exhibited two consecutive losses from 50-150° C., for a total loss of approximately 18%. The lost material was identified as chloroform by TGA-FTIR. This sample is therefore present, at least in part, as a chloroform solvate.

Form VI exhibited a 9-11% loss of mass from 80-140° C. The lost material was identified as chloroform by TGA-FTIR. This sample is therefore present, at least in part, as a chloroform solvate.

Form VII exhibited a 1-2% loss of mass from 80-150° C. The lost material was identified as methyl alcohol by TGA-FTIR. This sample is therefore present, at least in part, as a methyl alcohol solvate.

Form VIII exhibited losses of mass of 1.4% from 40° C. to 130° C. and approximately 2% from 30-100° C. The lost material was identified as water by TGA-FTIR, thus indicating that Form VIII is a hydrated crystalline form.

Form IX exhibited no significant loss of mass prior to sublimation, which occurred above approximately 135° C.

No TGA data was collected for Form X. Single crystal x-ray structure indicated a 1:1 molar ratio between levorphanol base and water. Form X is therefore a monohydrate crystalline form (approximately 4.2% water, by mass).

### Powder X-ray Diffraction

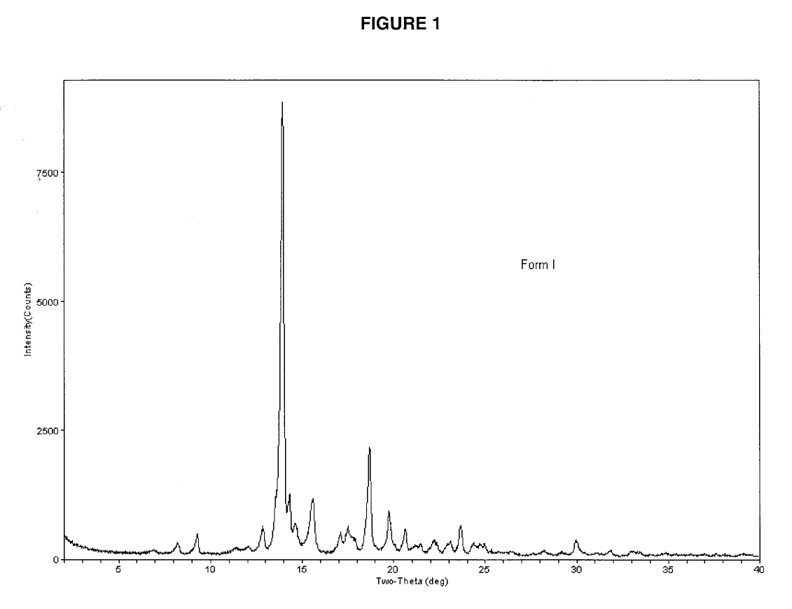

Analysis was conducted using a Siemens D500 X-ray Diffractometer. Samples of each of Forms I-X were uniformly crushed with a spatula edge, and placed on a quartz, zero-background holder. The following instrument parameters were utilized: Scan range −2.0 to 40.0°2θ, Step size—0.02°2θ, Scan time per step −1.0 seconds, Radiation source—copper Kα (1.5406 Å), X-ray tube power—40 kV/30 mA. The results are shown in FIGS. 1-10.

Additional information obtained from the powder X-ray diffraction analysis of Forms I-X is set forth in Tables 2-11. Crystalline levorphanol forms having at least four of the peaks indicated by an asterix (±0.2 deg 2θ) within any one of Tables 2-11 are preferred embodiments of the invention. More preferable are forms having at least eight of the peaks that are indicated by an asterix (±0.2 deg 2θ). Even more preferable are forms having at least ten of the peaks that are indicated by an asterix (±0.2 deg 2θ). Most preferable are forms having all of the peaks that are indicated by an asterix (±0.2 deg 2θ).

TABLE 2
Peak Search Report (Form I)

| 2-Theta | d(Å) | Height | H % | Area | A % |
|---|---|---|---|---|---|
| 6.9 | 12.7 | 78 | 0.9 | 1398 | 1.1 |
| *8.2 | 10.7 | 213 | 2.5 | 2820 | 2.3 |
| *9.3 | 9.5 | 377 | 4.4 | 4484 | 3.7 |
| 11.4 | 7.8 | 86 | 1.0 | 2306 | 1.9 |
| 12.0 | 7.3 | 82 | 1.0 | 1863 | 1.5 |
| *12.9 | 6.9 | 416 | 4.8 | 4315 | 3.5 |
| *14.0 | 6.3 | 8579 | 100.0 | 122590 | 100.0 |
| *14.4 | 6.2 | 944 | 11.0 | 17306 | 14.1 |
| 14.7 | 6.0 | 237 | 2.8 | 1730 | 1.4 |
| *15.6 | 5.7 | 1044 | 12.2 | 20594 | 16.8 |
| 17.1 | 5.2 | 365 | 4.3 | 6732 | 5.5 |
| 17.5 | 5.1 | 467 | 5.4 | 12600 | 10.3 |
| 17.9 | 5.0 | 224 | 2.6 | 6302 | 5.1 |
| *18.7 | 4.7 | 1962 | 22.9 | 24730 | 20.2 |
| *19.8 | 4.5 | 741 | 8.6 | 9502 | 7.8 |
| *20.7 | 4.3 | 402 | 4.7 | 4592 | 3.7 |
| 21.2 | 4.2 | 124 | 1.4 | 2576 | 2.1 |
| 21.5 | 4.1 | 138 | 1.6 | 2087 | 1.7 |
| 22.2 | 4.0 | 221 | 2.6 | 3512 | 2.9 |
| 23.1 | 3.8 | 210 | 2.4 | 3486 | 2.8 |
| *23.7 | 3.8 | 526 | 6.1 | 6142 | 5.0 |
| 24.4 | 3.6 | 185 | 2.2 | 3542 | 2.9 |
| 25.0 | 3.6 | 176 | 2.0 | 5364 | 4.4 |
| 28.2 | 3.2 | 99 | 1.2 | 2111 | 1.7 |
| *30.0 | 3.0 | 279 | 3.3 | 4493 | 3.7 |
| 31.8 | 2.8 | 95 | 1.1 | 925 | 0.8 |
| 33.0 | 2.7 | 87 | 1.0 | 2556 | 2.1 |

TABLE 3
Peak Search Report (Form II)

| 2-Theta | d(Å) | Height | H % | Area | A % |
|---|---|---|---|---|---|
| 2.4 | 37.4 | 185 | 3.4 | 4930 | 6.4 |
| *9.8 | 9.0 | 2178 | 39.5 | 36141 | 47.2 |
| *10.5 | 8.4 | 2528 | 45.8 | 34285 | 44.7 |
| *12.5 | 7.1 | 5514 | 100.0 | 76636 | 100.0 |
| 15.2 | 5.8 | 62 | 1.1 | 833 | 1.1 |
| *17.0 | 5.2 | 850 | 15.4 | 12323 | 16.1 |
| *17.5 | 5.1 | 974 | 17.7 | 11846 | 15.5 |
| *18.3 | 4.8 | 814 | 14.8 | 17089 | 22.3 |
| *18.7 | 4.7 | 2095 | 38.0 | 27324 | 35.7 |
| 19.6 | 4.5 | 74 | 1.3 | 907 | 1.2 |
| 20.0 | 4.4 | 78 | 1.4 | 872 | 1.1 |
| *21.2 | 4.2 | 1085 | 19.7 | 14431 | 18.8 |
| *21.7 | 4.1 | 476 | 8.6 | 5683 | 7.4 |
| *23.2 | 3.8 | 1176 | 21.3 | 17511 | 22.8 |
| 25.1 | 3.6 | 66 | 1.2 | 800 | 1.0 |
| 25.8 | 3.5 | 88 | 1.6 | 2321 | 3.0 |
| 26.2 | 3.4 | 214 | 3.9 | 4425 | 5.8 |
| *26.4 | 3.4 | 457 | 8.3 | 7876 | 10.3 |
| *27.3 | 3.3 | 454 | 8.2 | 5436 | 7.1 |
| 28.7 | 3.1 | 245 | 4.4 | 3113 | 4.1 |
| 29.7 | 3.0 | 88 | 1.6 | 1022 | 1.3 |
| 30.5 | 2.9 | 112 | 2.0 | 1878 | 2.5 |
| *32.7 | 2.7 | 314 | 5.7 | 3879 | 5.1 |
| 34.4 | 2.6 | 41 | 0.8 | 783 | 1.0 |
| 35.4 | 2.5 | 85 | 1.5 | 897 | 1.2 |
| 35.9 | 2.5 | 98 | 1.8 | 1237 | 1.6 |
| 36.6 | 2.5 | 62 | 1.1 | 812 | 1.1 |
| 37.9 | 2.4 | 137 | 2.5 | 1917 | 2.5 |

TABLE 4
Peak Search Report (Form III)

| 2-Theta | d(Å) | Height | H % | Area | A % |
|---|---|---|---|---|---|
| *5.9 | 14.9 | 988 | 6.3 | 17896 | 7.0 |
| *9.7 | 9.1 | 368 | 2.4 | 5954 | 2.3 |
| *11.9 | 7.4 | 15569 | 100.0 | 256021 | 100.0 |
| 13.4 | 6.6 | 284 | 1.8 | 4429 | 1.7 |
| 14.2 | 6.2 | 590 | 3.8 | 14873 | 5.8 |
| *15.4 | 5.7 | 3233 | 20.8 | 46941 | 18.3 |
| 16.6 | 5.4 | 106 | 0.7 | 1922 | 0.8 |
| 18.4 | 4.8 | 268 | 1.7 | 3737 | 1.5 |
| *19.6 | 4.5 | 4159 | 26.7 | 69539 | 27.2 |
| *20.0 | 4.5 | 479 | 3.1 | 15928 | 6.2 |
| 21.4 | 4.2 | 192 | 1.2 | 6510 | 2.5 |
| 22.8 | 3.9 | 377 | 2.4 | 9341 | 3.6 |
| 23.8 | 3.7 | 107 | 0.7 | 1137 | 0.4 |
| *25.3 | 3.5 | 995 | 6.4 | 14689 | 5.7 |
| *26.2 | 3.4 | 828 | 5.3 | 17923 | 7.0 |
| 30.1 | 3.0 | 141 | 0.9 | 2749 | 1.1 |
| 31.1 | 2.9 | 106 | 0.7 | 1612 | 0.6 |
| 31.9 | 2.8 | 56 | 0.4 | 1236 | 0.5 |
| 33.6 | 2.7 | 164 | 1.1 | 1637 | 0.6 |
| 34.1 | 2.6 | 112 | 0.7 | 4000 | 1.6 |
| 36.3 | 2.5 | 246 | 1.6 | 3677 | 1.4 |
| 37.2 | 2.4 | 47 | 0.3 | 753 | 0.3 |
| 38.2 | 2.4 | 68 | 0.4 | 1331 | 0.5 |

TABLE 5
Peak Search Report (Form IV)

| 2-Theta | d(Å) | Height | H % | Area | A % |
|---|---|---|---|---|---|
| *7.4 | 11.9 | 144 | 3.5 | 2953 | 4.8 |
| *9.6 | 9.2 | 208 | 5.0 | 2294 | 3.7 |
| *12.4 | 7.1 | 2140 | 51.7 | 27107 | 44.0 |
| *13.6 | 6.5 | 2185 | 52.8 | 31499 | 51.1 |
| 14.0 | 6.3 | 99 | 2.4 | 748 | 1.2 |
| *14.9 | 6.0 | 1942 | 46.9 | 27853 | 45.2 |
| *15.5 | 5.7 | 1119 | 27.0 | 13037 | 21.1 |
| *16.8 | 5.3 | 1692 | 40.9 | 20633 | 33.5 |
| *18.8 | 4.7 | 565 | 13.7 | 6303 | 10.2 |
| *19.7 | 4.5 | 4140 | 100.0 | 61653 | 100.0 |
| 20.1 | 4.4 | 677 | 16.4 | 24980 | 40.5 |
| 21.0 | 4.2 | 144 | 3.5 | 1911 | 3.1 |
| 22.0 | 4.0 | 97 | 2.3 | 1098 | 1.8 |
| 22.6 | 3.9 | 166 | 4.0 | 2506 | 4.1 |
| 23.4 | 3.8 | 62 | 1.5 | 686 | 1.1 |
| 24.3 | 3.7 | 152 | 3.7 | 2637 | 4.3 |
| 24.9 | 3.6 | 94 | 2.3 | 1819 | 2.9 |
| 25.6 | 3.5 | 87 | 2.1 | 1150 | 1.9 |
| *26.5 | 3.4 | 953 | 23.0 | 15253 | 24.7 |
| 28.1 | 3.2 | 91 | 2.2 | 1267 | 2.1 |
| 28.6 | 3.1 | 33 | 0.8 | 654 | 1.1 |
| 29.2 | 3.1 | 52 | 1.3 | 686 | 1.1 |
| 30.1 | 3.0 | 49 | 1.2 | 560 | 0.9 |
| 31.0 | 2.9 | 46 | 1.1 | 746 | 1.2 |
| 31.8 | 2.8 | 33 | 0.8 | 704 | 1.1 |
| 32.6 | 2.7 | 53 | 1.3 | 1451 | 2.4 |
| 33.2 | 2.7 | 35 | 0.8 | 224 | 0.4 |
| *34.1 | 2.6 | 163 | 3.9 | 2201 | 3.6 |
| 35.0 | 2.6 | 76 | 1.8 | 986 | 1.6 |
| 37.8 | 2.4 | 127 | 3.1 | 1585 | 2.6 |
| 39.0 | 2.3 | 49 | 1.2 | 1170 | 1.9 |
| 39.5 | 2.3 | 36 | 0.9 | 732 | 1.2 |

TABLE 6
Peak Search Report (Form V)

| 2-Theta | d(Å) | Height | H % | Area | A % |
|---|---|---|---|---|---|
| *9.1 | 9.7 | 188 | 13.1 | 3386 | 9.4 |
| *10.3 | 8.6 | 709 | 49.5 | 14979 | 41.4 |
| *13.1 | 6.8 | 1433 | 100.0 | 36160 | 100.0 |
| *14.2 | 6.2 | 689 | 48.1 | 14066 | 38.9 |
| *16.5 | 5.4 | 781 | 54.5 | 23387 | 64.7 |
| *17.1 | 5.2 | 493 | 34.4 | 11823 | 32.7 |
| *18.7 | 4.8 | 832 | 58.0 | 23864 | 66.0 |
| 19.8 | 4.5 | 131 | 9.1 | 1270 | 3.5 |
| *20.5 | 4.3 | 369 | 25.7 | 10946 | 30.3 |
| *20.9 | 4.3 | 447 | 31.2 | 18941 | 52.4 |
| *21.3 | 4.2 | 310 | 21.6 | 4683 | 13.0 |
| *21.9 | 4.1 | 336 | 23.5 | 4364 | 12.1 |
| *22.9 | 3.9 | 263 | 18.4 | 5808 | 16.1 |
| *23.9 | 3.7 | 152 | 10.6 | 2168 | 6.0 |
| *25.0 | 3.6 | 241 | 16.8 | 5087 | 14.1 |
| 25.4 | 3.5 | 129 | 9.0 | 3108 | 8.6 |
| 26.9 | 3.3 | 74 | 5.2 | 1234 | 3.4 |
| *28.1 | 3.2 | 210 | 14.6 | 3943 | 10.9 |
| *29.8 | 3.0 | 129 | 9.0 | 1891 | 5.2 |
| *30.8 | 2.9 | 147 | 10.2 | 2569 | 7.1 |
| 31.8 | 2.8 | 83 | 5.8 | 784 | 2.2 |
| 32.8 | 2.7 | 85 | 5.9 | 1733 | 4.8 |
| 33.8 | 2.7 | 83 | 5.8 | 938 | 2.6 |
| 35.5 | 2.5 | 94 | 6.6 | 1818 | 5.0 |

TABLE 7
Peak Search Report (Form VI)

| 2-Theta | d(Å) | Height | H % | Area | A % |
|---|---|---|---|---|---|
| *10.9 | 8.1 | 191 | 8.1 | 2963 | 7.1 |
| *12.5 | 7.1 | 2354 | 100.0 | 38811 | 93.4 |
| *13.4 | 6.6 | 936 | 39.7 | 23874 | 57.4 |
| *13.8 | 6.4 | 919 | 39.0 | 26748 | 64.4 |
| *15.1 | 5.9 | 1029 | 43.7 | 41561 | 100.0 |
| *15.5 | 5.7 | 1551 | 65.9 | 28638 | 68.9 |
| 16.4 | 5.4 | 243 | 10.3 | 5249 | 12.6 |
| *16.9 | 5.2 | 1213 | 51.5 | 19767 | 47.6 |
| 18.2 | 4.9 | 117 | 5.0 | 2019 | 4.9 |
| *18.7 | 4.7 | 759 | 32.2 | 12569 | 30.2 |
| *20.0 | 4.5 | 1462 | 62.1 | 32867 | 79.1 |
| *20.4 | 4.4 | 1053 | 44.7 | 23964 | 57.7 |
| 21.3 | 4.2 | 437 | 18.5 | 8935 | 21.5 |
| 22.0 | 4.0 | 197 | 8.4 | 2426 | 5.8 |
| *22.8 | 3.9 | 477 | 20.2 | 14710 | 35.4 |
| *23.2 | 3.8 | 322 | 13.7 | 11274 | 27.1 |
| 24.5 | 3.6 | 234 | 9.9 | 5706 | 13.7 |
| 25.4 | 3.5 | 268 | 11.4 | 3151 | 7.6 |
| *26.7 | 3.3 | 597 | 25.4 | 11272 | 27.1 |
| *27.2 | 3.3 | 293 | 12.5 | 6154 | 14.8 |
| 28.0 | 3.2 | 185 | 7.9 | 2756 | 6.6 |
| 28.8 | 3.1 | 120 | 5.1 | 3087 | 7.4 |
| 30.0 | 3.0 | 105 | 4.5 | 2472 | 5.9 |
| 30.9 | 2.9 | 119 | 5.1 | 1847 | 4.4 |
| 31.8 | 2.8 | 96 | 4.1 | 1157 | 2.8 |
| 33.9 | 2.6 | 139 | 5.9 | 2609 | 6.3 |
| 34.7 | 2.6 | 109 | 4.6 | 2117 | 5.1 |
| 36.6 | 2.5 | 132 | 5.6 | 2147 | 5.2 |
| 37.5 | 2.4 | 199 | 8.5 | 4582 | 11.0 |

TABLE 8
Peak Search Report (VII)

| 2-Theta | d(Å) | Height | H % | Area | A % |
|---|---|---|---|---|---|
| 9.9 | 8.9 | 134 | 6.0 | 2035 | 3.5 |
| *10.8 | 8.2 | 492 | 22.0 | 7369 | 12.6 |
| *12.1 | 7.3 | 302 | 13.5 | 5031 | 8.6 |
| 12.4 | 7.1 | 299 | 13.3 | 4441 | 7.6 |
| *12.8 | 6.9 | 1301 | 58.0 | 16747 | 28.7 |
| *13.4 | 6.6 | 1542 | 68.8 | 27217 | 46.6 |
| 13.9 | 6.4 | 130 | 5.8 | 941 | 1.6 |
| *14.4 | 6.2 | 2241 | 100.0 | 58360 | 100.0 |
| *15.1 | 5.9 | 1261 | 56.2 | 20996 | 36.0 |
| 15.6 | 5.7 | 139 | 6.2 | 810 | 1.4 |
| *16.5 | 5.4 | 889 | 39.6 | 12958 | 22.2 |
| *17.1 | 5.2 | 1498 | 66.8 | 20647 | 35.4 |
| *17.3 | 5.1 | 943 | 42.1 | 13886 | 23.8 |
| 17.7 | 5.0 | 415 | 18.5 | 5055 | 8.7 |
| *18.1 | 4.9 | 1311 | 58.5 | 15742 | 27.0 |
| *19.5 | 4.5 | 1094 | 48.8 | 14015 | 24.0 |
| 20.1 | 4.4 | 187 | 8.4 | 3219 | 5.5 |
| 21.1 | 4.2 | 264 | 11.8 | 1255 | 2.2 |
| *21.5 | 4.1 | 609 | 27.2 | 7150 | 12.3 |
| *21.7 | 4.1 | 592 | 26.4 | 9063 | 15.5 |
| 22.0 | 4.0 | 349 | 15.6 | 3900 | 6.7 |
| 22.5 | 4.0 | 325 | 14.5 | 3150 | 5.4 |
| *23.9 | 3.7 | 425 | 19.0 | 6525 | 11.2 |
| 24.8 | 3.6 | 224 | 10.0 | 4776 | 8.2 |
| 26.0 | 3.4 | 186 | 8.3 | 3943 | 6.8 |
| 26.5 | 3.4 | 117 | 5.2 | 3700 | 6.3 |
| *27.3 | 3.3 | 336 | 15.0 | 2305 | 3.9 |
| 27.8 | 3.2 | 114 | 5.1 | 1296 | 2.2 |
| 28.5 | 3.1 | 172 | 7.7 | 1179 | 2.0 |
| 29.1 | 3.1 | 195 | 8.7 | 3662 | 6.3 |
| 29.3 | 3.1 | 113 | 5.1 | 2496 | 4.3 |
| 29.7 | 3.0 | 127 | 5.7 | 1750 | 3.0 |
| 30.7 | 2.9 | 188 | 8.4 | 1906 | 3.3 |

TABLE 9
Peak Search Report (Form VIII)

| 2-Theta | d(Å) | Height | H % | Area | A % |
|---|---|---|---|---|---|
| 2.3 | 39.1 | 365 | 17.3 | 7501 | 14.9 |
| 7.2 | 12.2 | 143 | 6.8 | 2786 | 5.5 |
| *10.0 | 8.9 | 269 | 12.8 | 5949 | 11.8 |
| 11.1 | 8.0 | 159 | 7.5 | 3228 | 6.4 |
| *11.6 | 7.7 | 469 | 22.2 | 11163 | 22.1 |
| *12.2 | 7.2 | 879 | 41.7 | 16402 | 32.5 |
| 12.8 | 6.9 | 270 | 12.8 | 3759 | 7.4 |
| *13.8 | 6.4 | 2107 | 100.0 | 50462 | 100.0 |
| *14.6 | 6.1 | 806 | 38.3 | 9747 | 19.3 |
| *15.4 | 5.7 | 2051 | 97.3 | 35826 | 71.0 |
| 16.7 | 5.3 | 269 | 12.8 | 7748 | 15.4 |
| 17.0 | 5.2 | 409 | 19.4 | 13554 | 26.9 |
| 17.7 | 5.0 | 245 | 11.6 | 3385 | 6.7 |
| 18.1 | 4.9 | 443 | 21.0 | 15145 | 30.0 |
| 18.5 | 4.8 | 264 | 12.5 | 4145 | 8.2 |
| 19.7 | 4.5 | 421 | 20.0 | 9666 | 19.2 |
| 20.6 | 4.3 | 341 | 16.2 | 14474 | 28.7 |
| *21.3 | 4.2 | 652 | 30.9 | 22842 | 45.3 |
| *23.2 | 3.8 | 403 | 19.1 | 8302 | 16.5 |
| 23.8 | 3.7 | 129 | 6.1 | 5806 | 11.5 |
| *24.3 | 3.7 | 264 | 12.5 | 4943 | 9.8 |
| *25.0 | 3.6 | 185 | 8.8 | 3268 | 6.5 |
| 26.6 | 3.4 | 123 | 5.9 | 1901 | 3.8 |
| 31.0 | 2.9 | 115 | 5.4 | 1904 | 3.8 |
| 33.2 | 2.7 | 88 | 4.2 | 2938 | 5.8 |
| 36.8 | 2.4 | 93 | 4.4 | 2576 | 5.1 |

TABLE 10
Peak Search Report (Form IX)

| 2-Theta | d(Å) | Height | H % | Area | A % |
|---|---|---|---|---|---|
| *8.4 | 10.6 | 157 | 8.5 | 2605 | 3.5 |
| *9.9 | 8.9 | 301 | 16.3 | 5336 | 7.2 |
| *11.8 | 7.5 | 462 | 25.1 | 13300 | 17.9 |
| *12.2 | 7.3 | 252 | 13.7 | 12396 | 16.7 |
| *14.1 | 6.3 | 1842 | 100.0 | 74284 | 100.0 |
| *15.7 | 5.6 | 1001 | 54.3 | 17620 | 23.7 |
| *16.7 | 5.3 | 446 | 24.2 | 7794 | 10.5 |
| *18.1 | 4.9 | 600 | 32.6 | 13035 | 17.5 |
| *19.7 | 4.5 | 312 | 17.0 | 7774 | 10.5 |
| 20.2 | 4.4 | 174 | 9.5 | 6193 | 8.3 |
| *21.3 | 4.2 | 451 | 24.5 | 17281 | 23.3 |
| *21.8 | 4.1 | 517 | 28.0 | 9316 | 12.5 |
| *22.6 | 3.9 | 147 | 8.0 | 5525 | 7.4 |
| *23.3 | 3.8 | 182 | 9.9 | 6811 | 9.2 |
| 24.9 | 3.6 | 77 | 4.2 | 1342 | 1.8 |

TABLE 11
Peak Search Report (Form X)

| 2-Theta | d(Å) | Height | H % | Area | A % |
|---|---|---|---|---|---|
| *11.3 | 7.8 | 1896 | 19.0 | 21374 | 18.7 |
| *12.1 | 7.3 | 9965 | 100.0 | 114057 | 100.0 |
| *14.3 | 6.2 | 2134 | 21.4 | 24577 | 21.5 |
| *17.3 | 5.1 | 5474 | 54.9 | 68283 | 59.9 |
| *19.8 | 4.5 | 1633 | 16.4 | 18339 | 16.1 |
| 20.3 | 4.4 | 359 | 3.6 | 8409 | 7.4 |
| *20.7 | 4.3 | 1808 | 18.1 | 18488 | 16.2 |
| 21.7 | 4.1 | 248 | 2.5 | 3616 | 3.2 |
| *22.9 | 3.9 | 898 | 9.0 | 11047 | 9.7 |
| *23.3 | 3.8 | 2049 | 20.6 | 21025 | 18.4 |
| *23.6 | 3.8 | 666 | 6.7 | 7641 | 6.7 |
| 24.3 | 3.7 | 429 | 4.3 | 11261 | 9.9 |
| *24.6 | 3.6 | 1169 | 11.7 | 14202 | 12.5 |
| *26.4 | 3.4 | 385 | 3.9 | 5057 | 4.4 |
| *28.5 | 3.1 | 397 | 4.0 | 4252 | 3.7 |
| *29.4 | 3.03 | 243 | 2.4 | 2269 | 2.0 |
| *30.5 | 2.9 | 203 | 2.0 | 2369 | 2.1 |
| *31.7 | 2.8 | 399 | 4.0 | 5150 | 4.5 |
| *34.6 | 2.6 | 317 | 3.2 | 4432 | 3.9 |
| *34.9 | 2.6 | 413 | 4.1 | 8563 | 7.5 |

### Pharmaceutical Compositions and Methods

In accordance with the present invention, these novel crystalline polymorphs of levorphanol may be prepared as pharmaceutical compositions that are particularly useful for the management of pain, particularly chronic and/or severe pain. Such compositions comprise one of the new polymorphic forms of levorphanol with pharmaceutically acceptable carriers and/or excipients that are known to those skilled in the art.

Preferably, these compositions are prepared as medicaments to be administered orally. Suitable forms for oral administration include tablets, compressed or coated pills, hard or gelatin capsules, sub-lingual tablets, syrups, and suspensions. While one of skill in the art will understand that dosages will vary according to indication, age of patient, etc., generally the polymorphic forms of levorphanol of the present invention will be administered from about 2 mg to about 4 mg every 4-6 hours, for a total of about 8 mg to about 24 mg per day, and preferably from about 12 mg to about 20 mg per day.

### CONCLUSION

Additional uses for the crystalline polymorphs described herein, as well as the compositions containing these polymorphs, and the methods of administering the compositions, are also envisioned. For example, and without limitation, the crystalline polymorphs and related pharmaceutical compositions of the present invention may also be useful in treating any conditions that may be alleviated by administration of opioid analgesics. The principles applied to the formation of the oral pharmaceutical compositions disclosed herein may also be applied to the formation of other delivery vehicles, such as sublingual, vaginal, rectal, etc.

While the present invention has been described for what are presently considered the preferred embodiments, the invention is not so limited. To the contrary, the invention is intended to cover various modifications and equivalent arrangements included within the spirit and scope of the detailed description provided above.

We claim:

1. A crystalline form of levorphanol characterized by an x-ray diffraction pattern having characteristic peaks expressed in degrees 2θ (±0.2°) at 8.2, 9.3, 12.9, 14.0,14.4, 15.6, 18.7, 19.8, 20.7, 23.7, and 30.0.

2. The crystalline form of levorphanol of claim 1 characterized by a powder x-ray diffraction spectrum substantially as shown in FIG. 1.

3. The crystalline form of levorphanol of claim 1 characterized by a melt/decompose temperature of approximately 200° C.

4. The crystalline form of levorphanol of claim 1 characterized by no loss of mass prior to sublimation above 135° C.

5. A crystalline form of levorphanol characterized by an x-ray diffraction pattern having characteristic peaks expressed in degrees 2θ (±0.2°) at 9.8, 10.5, 12.5, 17.0, 17.5, 18.3, 18.7, 21.2, 21.7, 23.2, 26.4, 27.3, 32.7.

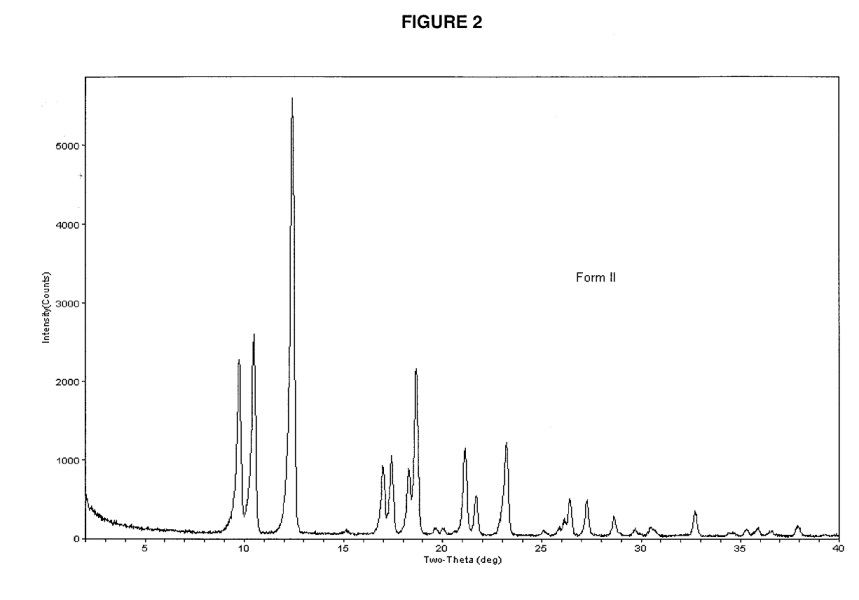

6. The crystalline form of levorphanol of claim 5 characterized by a powder x-ray diffraction spectrum substantially as shown in FIG. 2.

7. The crystalline form of levorphanol of claim 5 characterized by a melt/decompose temperature of approximately 200° C.

8. The crystalline form of levorphanol of claim 5 characterized by no loss of mass prior to sublimation above 135° C.

9. A crystalline acetonitrile solvated form of levorphanol characterized by an x-ray diffraction pattern having characteristic peaks expressed in degrees 2θ (±0.2°) at 5.9, 9.7, 11.9, 15.4, 19.6, 20.0, 25.3, 26.2.

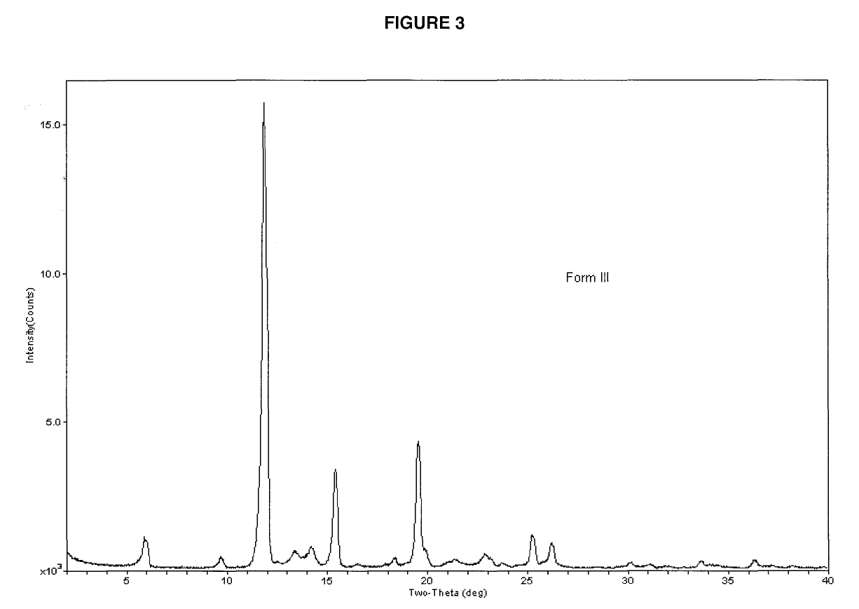

10. The crystalline form of levorphanol of claim 9 characterized by a powder x-ray diffraction spectrum substantially as shown in FIG. 3.

11. The crystalline form of levorphanol of claim 9 characterized by a melt/decompose temperature of approximately 200° C.

12. The crystalline form of levorphanol of claim 9 characterized by a loss of 6-7% acetonitrile from 80-145° C.

13. A crystalline methylene chloride solvated form of levorphanol characterized by an x-ray diffraction pattern having characteristic peaks expressed in degrees 2θ (±0.2°) at 7.4, 9.6, 12.4, 13.6, 14.9, 15.5, 16.8, 18.8, 19.7, 26.5, 34.1.

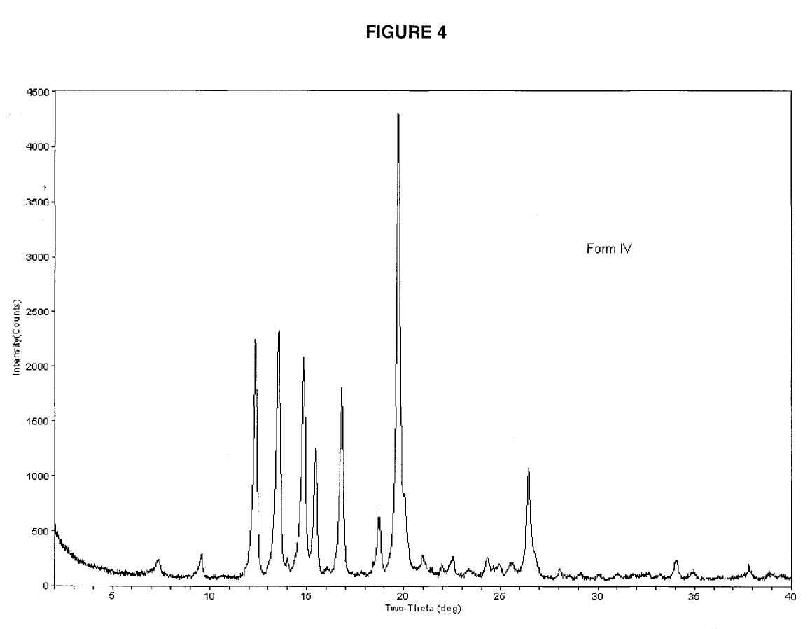

14. The crystalline form of levorphanol of claim 13 characterized by a powder x-ray diffraction spectrum substantially as shown in FIG. 4.

15. The crystalline form of levorphanol of claim 13 characterized by a melt/decompose temperature of approximately 200° C.

16. The crystalline form of levorphanol of claim 13 characterized by a loss of 7-8% methylene chloride from 110-140° C.

17. A crystalline chloroform solvated form of levorphanol characterized by an x-ray diffraction pattern having characteristic peaks expressed in degrees 2θ (±0.2°) at 9.1, 10.3, 13.1, 14.2, 16.5, 17.1, 18.7, 20.5, 20.9, 21.3, 21.9, 22.9, 23.9, 25.0, 28.1, 29.8, 30.8.

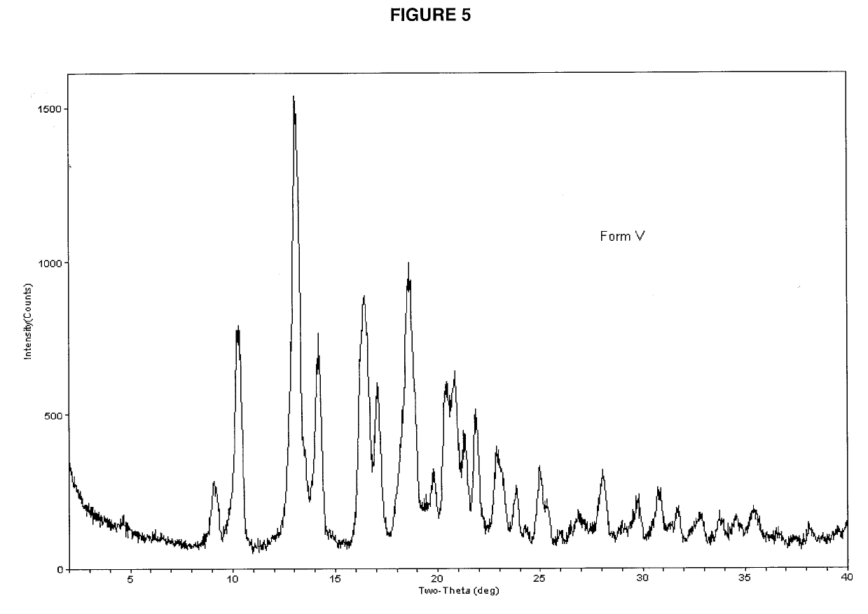

18. The crystalline form of levorphanol of claim 17 characterized by a powder x-ray diffraction spectrum substantially as shown in FIG. 5.

19. The crystalline form of levorphanol of claim 17 characterized by a melt/decompose temperature of approximately 200° C.

20. The crystalline form of levorphanol of claim 17 characterized by two consecutive losses of chloroform from 50-150° C., for a total loss of approximately 18%.

21. A crystalline chloroform solvated form of levorphanol characterized by an x-ray diffraction pattern having characteristic peaks expressed in degrees 2θ (±0.2°) at 10.9, 12.5, 13.4, 13.8, 15.1, 15.5, 16.9, 18.7, 20.0, 20.4, 22.8, 23.2, 26.7, 27.2.

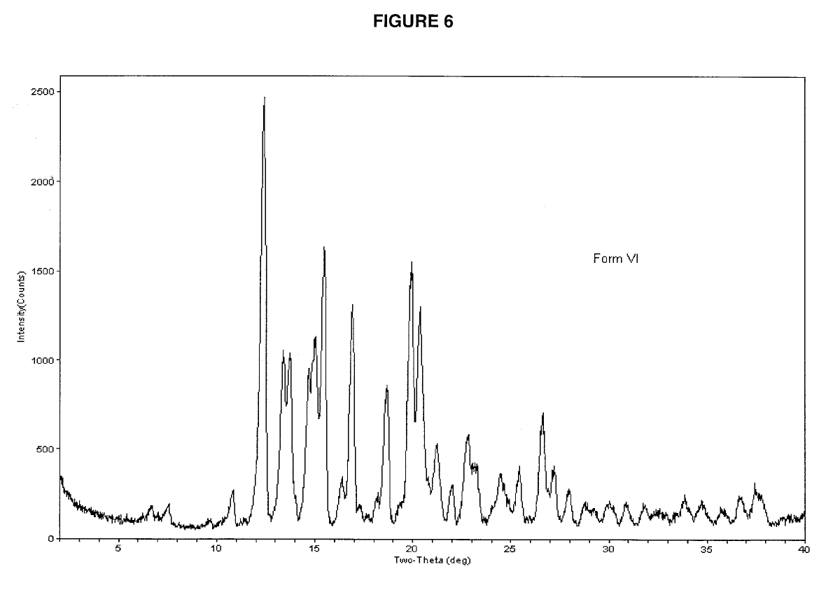

22. The crystalline form of levorphanol of claim 21 characterized by a powder x-ray diffraction spectrum substantially as shown in FIG. 6.

23. The crystalline form of levorphanol of claim 21 characterized by a melt/decompose temperature of approximately 200° C.

24. The crystalline form of levorphanol of claim 21 characterized by a 9-11% loss of chloroform from 80-140° C.

25. A crystalline methyl alcohol solvated form of levorphanol characterized by an x-ray diffraction pattern having characteristic peaks expressed in degrees 2θ (±0.2°) at 10.8, 12.1, 12.8, 13.4, 14.4, 15.1, 16.5, 17.1, 17.3, 18.1, 19.5, 21.5, 21.7, 23.9, 27.3.

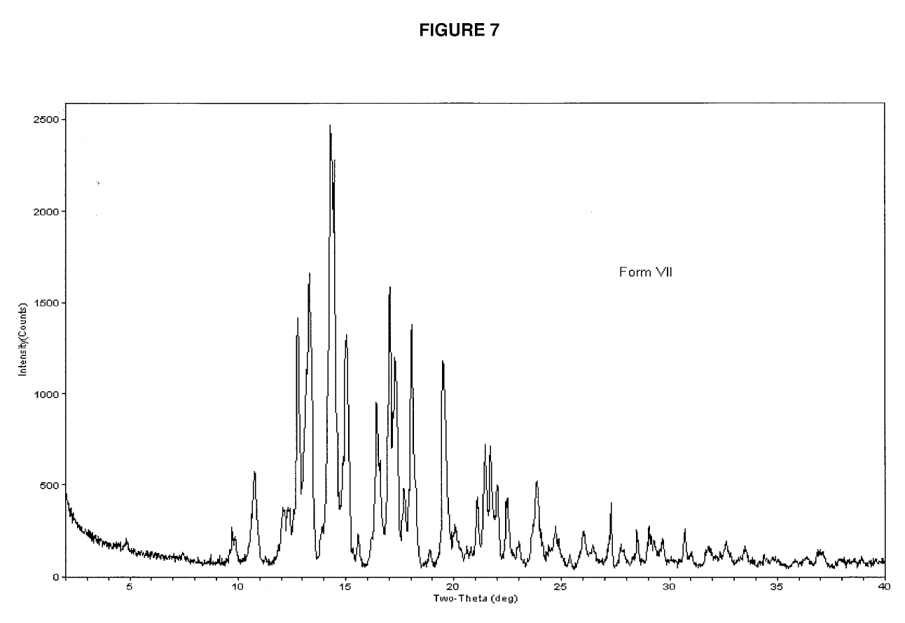

26. The crystalline form of levorphanol of claim 25 characterized by a powder x-ray diffraction spectrum substantially as shown in FIG. 7.

27. The crystalline form of levorphanol of claim 25 characterized by a melt/decompose temperature of from approximately 175-200° C.

28. The crystalline form of levorphanol of claim 25 characterized by a 1-2% loss of methyl alcohol from 80-150° C.

29. A crystalline hydrated form of levorphanol characterized by an x-ray diffraction pattern having characteristic peaks expressed in degrees 2θ (±0.2°) at 10.9, 11.6, 12.2, 13.8, 14.6, 15.4, 21.3, 23.2, 24.3, 25.0.

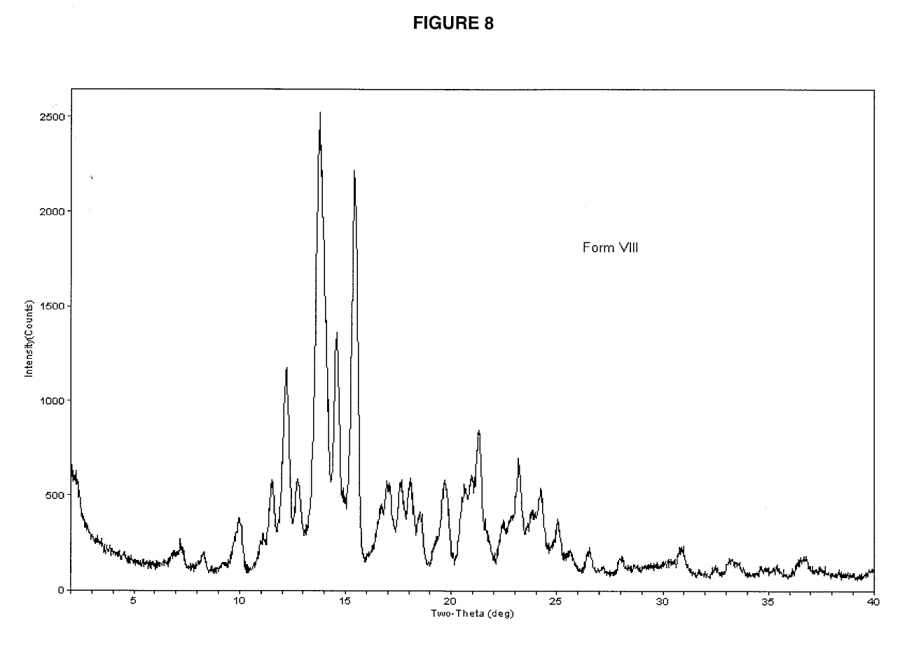

30. The crystalline form of levorphanol of claim 29 characterized by a powder x-ray diffraction spectrum substantially as shown in FIG. 8.

31. The crystalline form of levorphanol of claim 29 characterized by a melt/decompose temperature of approximately 200° C.

32. The crystalline form of levorphanol of claim 29 characterized by a loss of approximately 2% water from 30-100° C.

33. A crystalline non-hydrated form of levorphanol characterized by an x-ray diffraction pattern having characteristic peaks expressed in degrees 2θ (+0.2°) at 8.4, 9.9, 11.8, 12.2, 14.1, 15.7, 16.7, 18.1, 19.7, 21.3, 21.8, 22.6, 23.3.

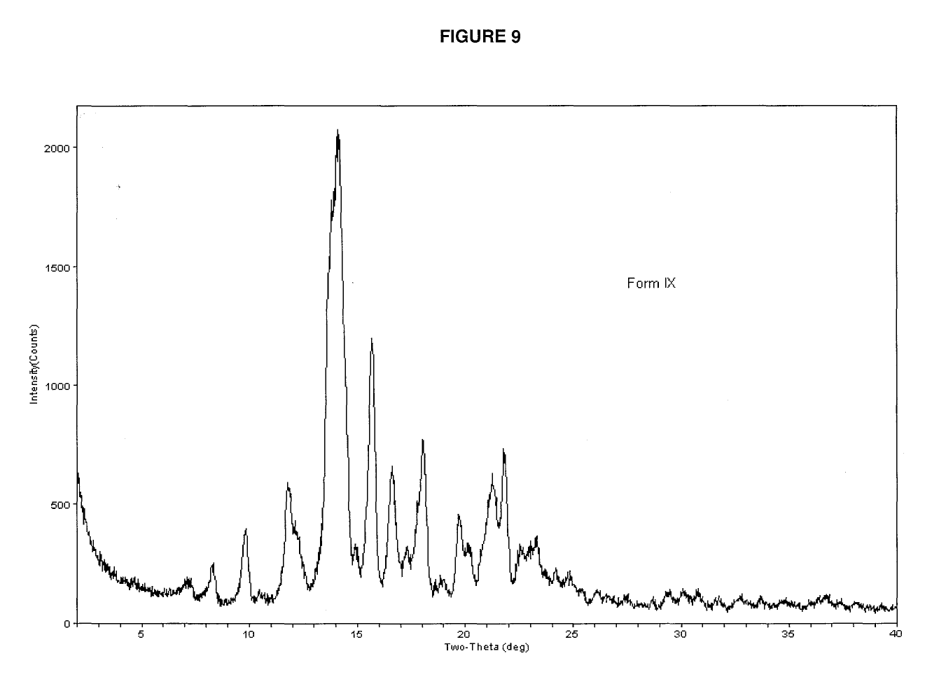

34. The crystalline form of levorphanol of claim 33 characterized by a powder x-ray diffraction spectrum substantially as shown in FIG. 9.

35. The crystalline form of levorphanol of claim 33 characterized by a melt/decompose temperature of approximately 200° C.

36. The crystalline form of levorphanol of claim 33 characterized by no loss of mass prior to sublimation above 135° C.

37. A crystalline monohydrated form of levorphanol characterized by an x-ray diffraction pattern having characteristic peaks expressed in degrees 2θ (±0.2°) at 11.3, 12.1, 14.3, 17.3, 19.8, 20.7, 22.9, 23.3, 23.6, 24.6, 26.4, 28.5, 29.4, 30.5, 31.7, 34.6, 34.9.

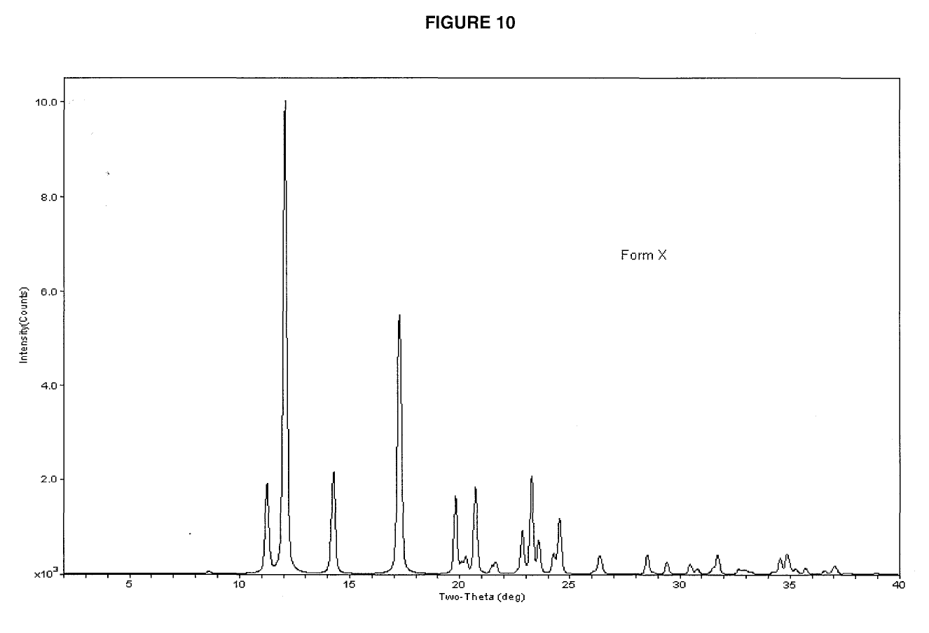

38. The crystalline form of levorphanol of claim 37 characterized by a powder x-ray diffraction spectrum substantially as shown in FIG. 10.

39. The crystalline form of levorphanol of claim 37 characterized by a 1:1 molar ratio between levorphanol base and water.

40. A pharmaceutical composition comprising a therapeutically-effective amount of the crystalline form of claim 1, and one or more pharmaceutically acceptable carriers, excipients or diluents thereof.

41. A method of relieving pain in a patient suffering therefrom, comprising the step of administering to said patient a therapeutically-effective amount of the crystalline form of claim 1.

42. A method of using the crystalline form as claimed in claim 1 in preparation of a pharmaceutical composition suitable for use in treating pain.
